(19) United States
(12) Patent Application Publication
(10) Pub. No.: US 2005/0226918 A1
(43) Pub. Date: Oct. 13, 2005

(54) Delivery system for insulin and other therapeutic agents

(76) Inventor: Stuart G. MacDonald, Pultneyville, NY (US)

Correspondence Address: HOWARD J. GREENWALD P.C., 349 W. COMMERCIAL STREET SUITE 2490, EAST ROCHESTER, NY, 14445-2408, US

(21) Appl. No.: 10/217,093

(22) Filed: Aug. 12, 2002

Related U.S. Application Data
(60) Provisional application No. 60/311,861, filed on Aug. 13, 2001.

(51) Int. Cl. A61F 13/00

(52) U.S. Cl. 424/449; 424/434

(57) ABSTRACT

An automated, non-implanted, closed-loop system for measurement of a biological material in an individual, determination of appropriate steady-state and bolus drug delivery response to the measured level of the biological material, and storage and delivery of a hormone, drug, or other chemical or biochemical therapeutic agent that serves to regulate or otherwise therapeutically react to the biological material.

### CROSS-REFERENCE TO RELATED PATENT APPLICATION

This application claims priority based upon applicant's provisional application 60/311,861, filed on Aug. 13, 2001.

### FIELD OF THE INVENTION

An automated, non-implanted, closed-loop system for delivery of insulin and other chemical therapeutic agents that provides efficient uptake, regulated chronic delivery, and patient convenience.

### BACKGROUND OF THE INVENTION

Various forms of delivery have been attempted specifically for synthetic insulin, for the control of diabetes. These forms include perenteral, self-injection, inhalation, implanted pumps, and the like. All of these forms of delivery suffer from one or more of the following problems: patient compliance (due to discomfort, inconvenience and embarrassment); low uptake efficiency; degradation due to enzymatic or metabolic breakdown; and unwillingness to accept an implantable device (that must be regularly refilled via skin puncture). One recent advance utilizes transport across the buccal membrane of the mouth; specialized formulations permit ready transport across the lining of the oral cavity and also protect the insulin itself from enzymatic breakdown due to enzymes in the saliva. U.S. Pat. No. 6,231,882 discloses a process for making a pharmaceutical composition suitable for delivery through mucosal membranes. Likewise, U.S. Pat. No. 6,221,378 discloses an alternate means to make a pharmaceutical composition suitable for delivery through mucosal membranes. U.S. Pat. No. 6,193,997 discloses an improved delivery system for the administration of large-molecule pharmaceuticals, e.g. peptidic drugs, vaccines and hormones. In particular it relates to pharmaceuticals which may be administered through the oral and nasal membranes, or by pulmonary access. Thus, by way of illustration and not limitation, one may use the methods described in U.S. Pat. No. 6,231,882, U.S. Pat. No. 6,221,378, and U.S. Pat. No. 6,193,997 individually or in combination, to create a pharmaceutical formulation optimized to deliver insulin or other large-molecule drugs via oral membranes. The entire disclosure of these patents is hereby incorporated into this specification.

A further limitation on the effectiveness of insulin therapy is the ability of the delivery system to maintain blood glucose concentration within a relatively narrow range. There is substantial clinical evidence that the long-term health of the diabetic individual, specifically the delay in onset of retinopathy, other nerve damage, loss of extremities, blindness, and even death, is directly related to the control of blood glucose concentration in the normal physiologic range, which is typically considered to be 70-110 mg/dl. Most forms of insulin delivery such as inhalation, self-injection, and more recent forms of delivery via the buccal lining of the mouth, result in delivery of a bolus of insulin; this acts to decrease blood glucose levels but does so in a relatively sudden manner and does not provide tight control of blood glucose concentration. An alternative that provides much better control is by infusion, either from an external source or from an implantable pumping device. The obvious limitation of an external source is the inconvenience, discomfort, and infection risk inherent in the infusion catheter. U.S. Pat. No. 5,957,890 discloses an implantable infusion pump with specialized features for maintaining constant flow rates. U.S. Pat. No. 6,248,093 discloses an improved pump is provided for controlled delivery of fluids wherein the pump includes a reservoir and a movable piston. Thus, by way of illustration and not limitation, one may use the methods described in U.S. Pat. No. 5,957,890 and U.S. Pat. No. 6,248,093, either separately or in combination; the entire disclosure of these patents is hereby incorporated into this specification.

U.S. Pat. No. 5,665,065 discloses a medication infusion device such as a programmable infusion pump that includes data input regarding a selected patient parameter such as a current blood glucose reading. The infusion device includes a controller responsive to this data input to develop a medication delivery protocol that can be implemented automatically. Thus by way of illustration and not limitation, one may use this method to control blood glucose concentration in response to fluctuations caused by diet and exercise; the entire disclosure of U.S. Pat. No. 5,665,065 is hereby incorporated into this specification.

Existing systems intended for the control of blood glucose in a diabetic individual by administration of insulin all have limitations as described above. It is the object of this invention to apply drug formulation and encapsulation technology, a miniaturized device comprising measurement, control, and pumping functions, and a novel method of applying drug to the buccal lining of the mouth, in order to create an insulin delivery system that overcomes the limitations of all prior forms of delivery.

By extension, this method may be applied to a large number of drugs that have similar limitations in biochemical compatibility or require chronic dosing that is either inconvenient, uncomfortable, or requiring an undesirable invasive procedure.

### SUMMARY OF THE INVENTION

In accordance with this invention, there is provided a drug delivery system which comprises means for measuring a biological material in an individual, means for determining appropriate steady-state and bolus drug delivery response to the measured level of said biological material, means for storing a hormone, drug, or other chemical or biochemical agent that serves to regulate or otherwise therapeutically react to said biological material, means for efficiently delivering said agent via one or more of the individual's mucosal membranes, means for providing algorithmic control, power, and recharging of the supply of said agent, and means for communication with an external device associated with functions such as status indications, alerts, long-term recordings, reprogramming, recalibration, or communication with said individual or individual's physician.

### BRIEF DESCRIPTION OF THE DRAWINGS

The invention will be described by reference to the specification and to the following drawings, in which like numerals refer to like elements, and in which:

FIG. 1 is a generalized diagram of the components of the system;

FIG. 2 is a flow diagram of the function of the system;

FIG. 3 is a schematic of one preferred assembly of the invention for obtaining measurements of the concentration of the biological material to be regulated;

FIG. 4 is a schematic of one preferred assembly for the application of said agent to the buccal lining of the mouth;

FIG. 5 is a schematic of one preferred assembly for a replenishable supply of said agent;

FIG. 6 is a schematic of a second preferred assembly for a replenishable supply of said agent;

FIG. 7 is a schematic of one preferred assembly and attachment of the overall system; and

FIG. 8 is a schematic of a second preferred assembly and attachment of the overall system.

### DESCRIPTION OF THE PREFERRED EMBODIMENTS

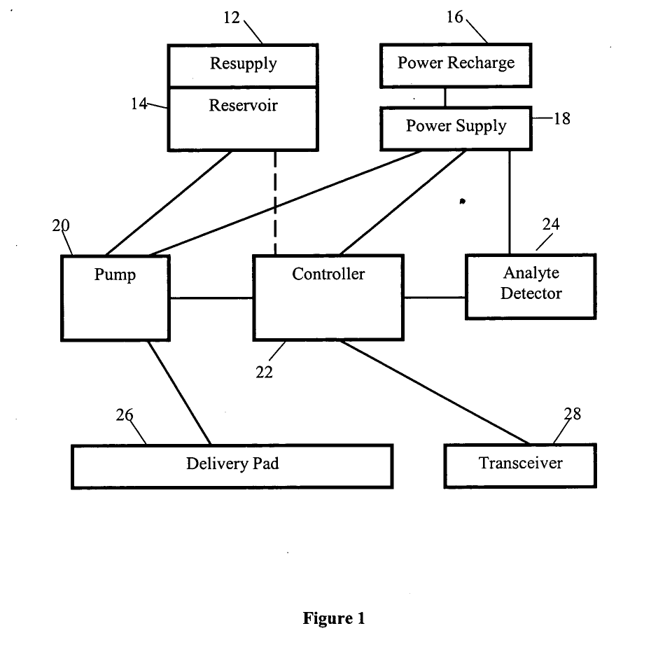

FIG. 1 is a diagram of the components of one preferred embodiment of a system that measures the concentration of a biological material in an individual, computes an amount of a biochemical or chemical agent that should be delivered to said individual based on an algorithm that uses a baseline steady state delivery rate and alters that rate based on said measurements, delivers said agent to the individual, and provides for periodic replenishment of the agent, for periodic recharging of the power source, and for bi-directional communication with the individual or a physician.

One preferred embodiment of this invention involves the measurement of blood glucose and the controlled delivery of insulin to a diabetic individual, in a manner that is more convenient and more stable than with previous systems. However, the steps and components disclosed herein can be used in similar manner with a wide range of disease states.

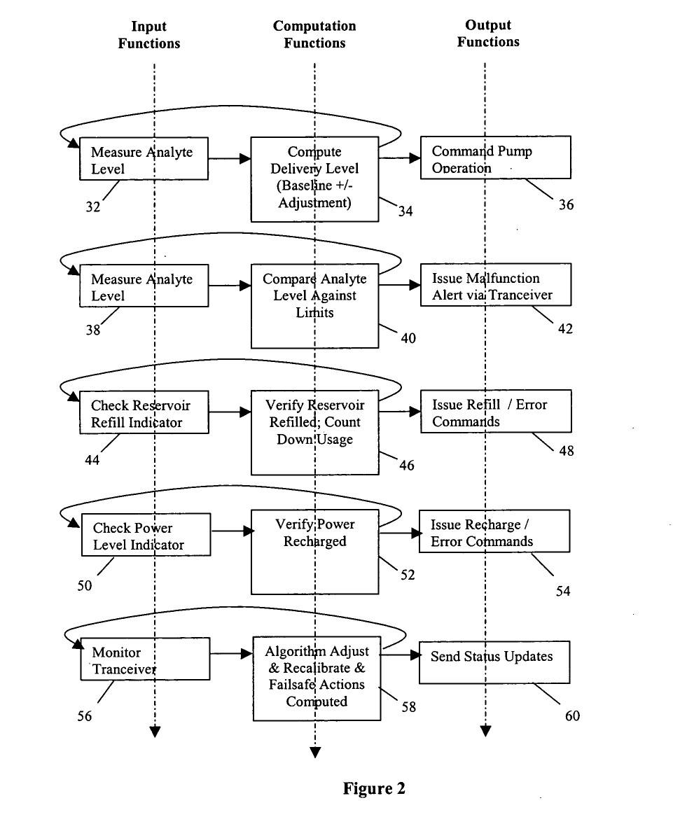

FIG. 2 is a flow diagram of the various functions of this system. Steps 32, 38, 44, 50, and 56 are input functions, providing measurements of an analyte of interest, either for purposes of adjusting the baseline delivery level as in step 32, or for purposes of alerting the individual or physician of an analyte concentration that is far outside the normal range and may represent a hazard to the individual. An example of this, and in one of the preferred embodiment, is the dosing of insulin in diabetic patients, and is used to regulate the level of blood glucose. Glucose measurements made as part of step 32 are intended to modestly adjust insulin levels after food is ingested in order to more closely regulate glucose level, and the same periodic glucose measurements are used in step 38 to provide a timely alert if and when the blood glucose level becomes too high (hyperglycemia) or too low (hypoglycemia).

Step 44 is a check function to determine whether the individual has properly resupplied the insulin reservoir; this is accomplished by an electrical contact that is closed when the resupply container is attached to the reservoir during resupply.

Step 50 is another check function to determine whether the individual has properly recharged the power source; in one embodiment this is accomplished by rapid recharging of a miniature electrical battery, but other energy storage means and alternate recharging means may be used.

Step 56 is a periodic communication with the bi-directional transceiver that will be later discussed as one of the system components. This communication is used to respond to changes in the resident computational algorithm, to send information relating to the long-term control of blood glucose, to alert the individual to recharge the reservoir and power source, or to send status alerts based on deviations from the normal control range of blood glucose.

Referring again to FIG. 2, steps 34, 40, 46, 52, and 58 are computational functions. Unlike the input functions and output functions also described in FIG. 2, the computational functions are all closely interrelated, thus there is a single programmed algorithm that has five basic functional responsibilities described in the aforementioned steps. To those skilled in the art, step 34 will be familiar as the computation of the desired rate of insulin dosing in real time. Implanted insulin delivery devices, among them those sold by the Minimed Company, are often resisted by diabetic individuals due to the required surgery and the uncomfortable process of recharging the implanted insulin reservoir. However, as previously discussed, there is considerable clinical evidence that by maintaining a very stable blood glucose level (˜70 to ˜110 mg/dl), and as indicated by the longer-term stability of the concentration of glycated hemoglobin in the blood, the long-term health of the individual can be greatly enhanced. Thus the best approach to insulin delivery is to deliver a baseline amount continuously, and to adjust that amount when food is ingested, preferably by measuring actual blood glucose and using a computer algorithm to adjust the insulin level in an optimal manner that does not over-react or under-react to short-term variation in glucose concentration. In contrast, most existing approaches to insulin dosing (self-injection, perenteral, and inhalers) provide a bolus of insulin; this approach will result in poorer control of glucose level, and poorer health outcomes for diabetic individuals.

Step 40 is very similar to step 34, but the information is used to alert the individual and/or the physician in cases where the blood glucose level becomes either dangerously high or low in spite of the operation of this system. Step 40 also serves as one of several self-monitoring checks the system regularly conducts on its own operation.

Step 46 is the computational process involved in verifying that the individual properly resupplied the insulin reservoir. If resupply is either delayed or done improperly an alert is sent to the individual.

Step 52 is the computational process involved in verifying that the individual properly recharged the power source. In the case of a rechargeable electric battery, simple measurements of voltage and current over time, along with the known discharge characteristics of the battery, will provide accurate information.

Step 58 is the computational process relating to changes in the overall algorithm or system-level failures. One method of system change is the process of accepting an external command and updating the algorithm based on physician input relating to a change in therapy. Another method of system change is the process of periodically calibrating the system by commanding specific changes in insulin delivery, monitoring the resulting physiological response, and adjusting algorithm parameters in order to compensate for the individual's unique physiological response, or minor changes in system performance, or both. A further method of system change is in response to system-level failures, either chronic or acute. Various alerts may be sent to the individual and/or the physician, and in the extreme, a failsafe shutdown procedure can be initiated.

Referring once again to FIG. 2, steps 36, 42, 48, 54, and 60 are output functions. Step 36 involves the actuation of a miniature pump that will be described later. The pump will be described in more detail, but can be a traditional piston pump, a miniature diaphragm pump, a peristaltic pump, a miniature dispenser similar to an ink-jet print head, or any of a wide variety of miniature fluid dispense devices. The applicator pad-associated with the pump and which provides the advantage of near-100% insulin uptake in the bloodstream will also be discussed later. Steps 42, 48, 54, and 60 all involve commands sent from the controller to the transceiver, for various reasons. The transceiver will be further described later, but can be any of a variety of miniature electronic devices such as those commonly referred to as ‘blue tooth’ or an acoustic transmitter/receiver comprised of piezoelectric material, an optical transmitter/receiver operating in the near infra-red, or any alternative such as those in common use in communications.

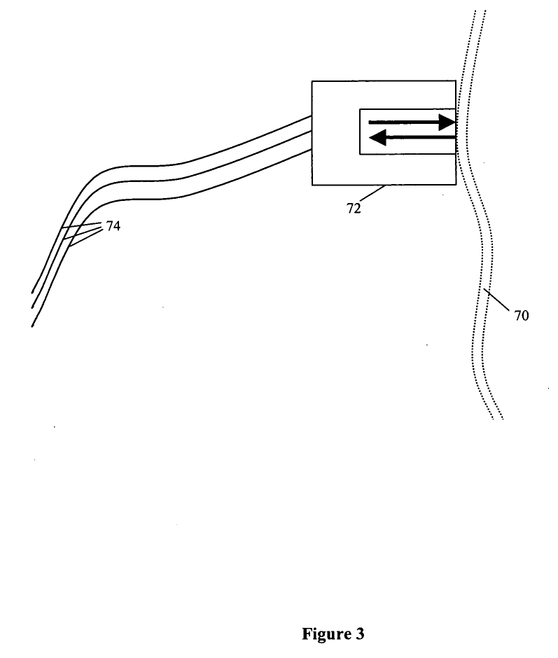

FIG. 3 is a schematic of one preferred means to measure blood glucose. It is well known in the art that by measuring between approximately 2 and 10 discrete wavelengths of light, typically in the near infra-red (NIR) region (from ˜700 nm to 3000 nm), determining the ratio of light at said discrete wavelengths, and applying an algorithm that uses first-derivative or second-derivative techniques, an accurate concentration measurement of an analyte such as glucose can be made in the presence of variable and unknown concentrations of other analytes that my interfere with the accuracy of other traditional techniques such as single-wavelength optical measurements. While much research has been done and many diagnostic companies have attempted this type of NIR measurement, variability in skin pigment and other factors such as light scatter and low levels of capillary blood near the skin surface have prevented an acceptably accurate and stable diagnostic. In FIG. 3 a near-infrared detector 72 is attached to the system controller and power supply via electrical conductors 74, and measurements are made of blood glucose in the buccal lining of the mouth. This method eliminates much of the effects of skin pigment and provides an adequately accurate signal for the purpose of this invention.

In another embodiment of this invention, conductors 74 may be optical rather than electrical, providing for a degree of compatibility and safety in the presence of radio frequency fields associated with magnetic resonance imaging (MRI). In still another embodiment of the present invention, detector 72 is used to measure the glucose concentration in saliva rather than in capillary blood; in that embodiment (not shown) the detector 72 has an additional capillary space at its distal end to permit access to saliva for NIR measurement.

In a further embodiment of this invention (not shown) the measurement of glucose concentration may be made using a semiconductor sensor in contact with saliva or in contact with the buccal lining. One method typically used incorporates the selective action of glucose oxidase (GOD) upon glucose to generate free electrons that create a signal in a chemfet or other semiconductor sensor.

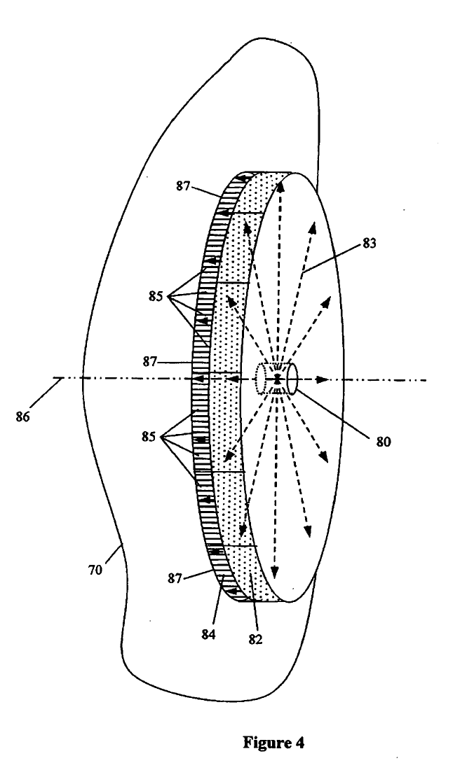

FIG. 4 is a schematic diagram of one preferred embodiment of a delivery pad that may be used to deliver insulin to the buccal lining of the mouth. The pad is comprised of a first chamber 82 that evenly distributes insulin that is pumped in from a tube at port 80 by way of capillary flow, said capillary distribution having a radial direction perpendicular to axis 86. The second chamber 84 is devised of capillary channels parallel to axis 86, and having capillary structure of higher capillary pressure than that in the radial-distribution first chamber 82. Thus the insulin delivered from the pump via port 80 is quickly and evenly across the pad in chamber 82, thence rapidly transported in chamber 84 to the interface between the chamber 84 and the buccal lining 70. This embodiment proves for highly efficient uptake of the insulin as found in the case of self-injection or implanted insulin pumps, and in contrast with the low uptake levels resulting from inhalation or perenteral delivery. The size and geometry of the delivery pad is designed to provide sufficient area to avoid diffusion limits to insulin uptake, and to provide a small, soft, and conformable component that is comfortable in the mouth.

Another embodiment (not shown) utilizes direct spraying of the desired amount of insulin via an array of ejection ports similar in nature and operation to typical ink-jet print heads.

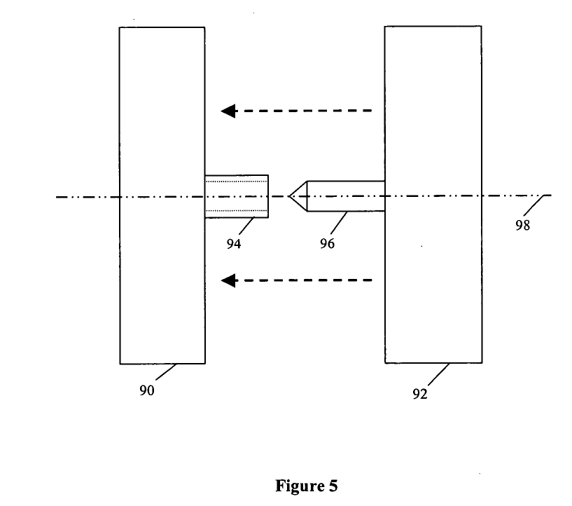

FIG. 5 illustrates one preferred embodiment for refilling the insulin reservoir 90. In this embodiment a disposable supply container 92, having a delivery probe 96, is inserted along axis 98 into port 94 of the reservoir 90. Refilling is achieved by removal and replacement of the nearly-empty supply container 92, which is attached to reservoir 90 by small detent features molded into the internal surface of port 94 and onto the exterior of probe 96. Port 94 contains a one-way valve that prevents backflow of insulin during the period when supply container 92 us detached. As previously described the amount of material remaining in the reservoir is known by the controller, and the alert to the diabetic individual to replace the nearly-empty supply container 92 is made with sufficient warning so that the supply is never exhausted. The highly efficient uptake of insulin and the lack of need for gas or other drivers in this pump-driven system result in a reservoir 90 size that is easily and comfortably retained in a small space in the individual's mouth.

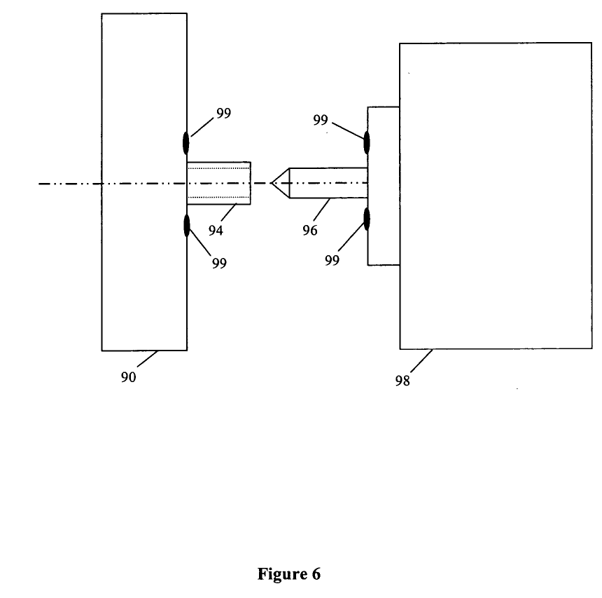

FIG. 6 illustrates a second preferred embodiment for refilling insulin reservoir 90. Port 94 contains a one-way valve that prevents backflow as in the previous embodiment. Resupply container 98 is a relatively larger, pressure-driven container that is applied to port 94 long enough for transfer of insulin from it into the reservoir 90. Contacts 99 are used for three purposes; indication of the fact that the resupply was performed, measurement of the duration of the resupply process to verify the resupply container 98 was in place long enough to complete the process, and also to permit recharging of the power source via electrical leads not shown.

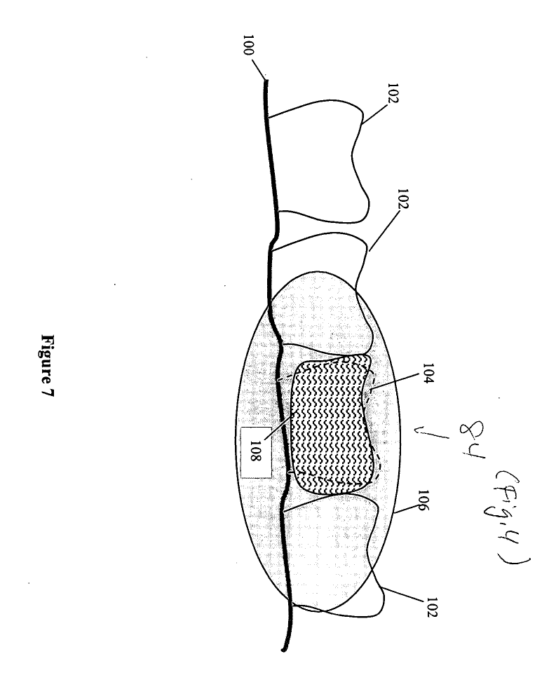

FIG. 7 illustrates one preferred embodiment of the placement and attachment of the overall system. Natural or false teeth 102 are shown along with the gum line 100, and a tooth 104 that has been recently extracted for dental health reasons or extracted to provide space to install the system of this invention. In one embodiment the system may be sufficiently miniaturized so as to comprise capture element 108 alone, and in another, it is still small but comprises both capture element 108 and main body 106. A further advantage of this last embodiment is that body 106 can on its exterior surface include the delivery surface of delivery pad chamber 84, previously described in FIG. 4. This device may be readily removed from the mouth for purposes of refilling and cleaning, and for the purpose of dental prophylaxis.

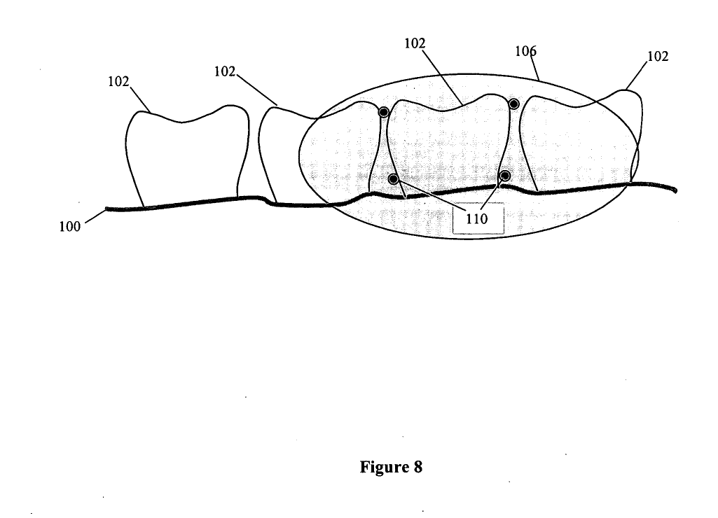

FIG. 8 illustrates another preferred embodiment of the placement and attachment of the overall system. In this embodiment none of the natural or false teeth 102 are disturbed. A set of flexible capture pins 110 are used to retain the device 106 in place, and contact with the buccal lining is achieved either by direct contact with the surface of delivery pad chamber 84 as in the previous embodiment, or by contact with a delivery pad chamber 84 that is remotely connected to the device 106 via a small tube (not shown). This device may also be readily removed from the mouth for purposes of refilling and cleaning, and for the purpose of dental prophylaxis.

Other embodiments of this invention, not described in detail, involve similar delivery means for chemical, drug, or hormone therapy via mucosal membranes at alternate sites on the individual's body, such as the nasal cavity or the vaginal cavity.

It is to be understood that the aforementioned description is illustrative only and that changes can be made in the apparatus, in the ingredients and materials, and in the sequence and combination of process steps, as well as in other aspects of the invention discussed herein, without departing from the scope of the invention as defined in the following claims.

1. A delivery system for a therapeutic agent, comprising means for continuously administering said therapeutic agent across a mucosal membrane, said means comprising a delivery pad having at an interface a first capillary chamber in contact with said mucosal membrane.

2. The delivery system as recited in claim 1, further comprising means for delivering said therapeutic agent to said mucosal membrane via a delivery pad comprising-one or more capillary chambers in contact with said mucosal membrane wherein said first capillary chamber composes capillary channels perpendicular to said interface.

3. The delivery system as recited in claim 1, further comprising means for delivering said therapeutic agent to said mucosal membrane via an array of miniature fluid jets.

4. The delivery system as recited in claim 1, further comprising a power source, a reservoir of therapeutic agents, means for recharging said power source, and means for refilling said reservoir of therapeutic agents.

5. The delivery system as recited in claim 1, further comprising means for bidirectional information and command transfer to an external device.

6. The delivery system as recited in claim 1, further comprising means for affixing said system in a mouth of a living organism.

7. A delivery system for a therapeutic agent, comprising means for monitoring the level of an analyte in a living body, means for computing an appropriate baseline and adjusted continuous administration rate for said therapeutic agent, and means for administering said therapeutic agent across a mucosal membrane.

8. The delivery system as recited in claim 7, further comprising means for delivering said therapeutic agent to said mucosal membrane via a delivery pad comprising one or more capillary chambers in contact with said mucosal membrane.

9. The delivery system as recited in claim 7, further comprising means for delivering said therapeutic agent to said mucosal membrane via an array of miniature fluid jets.

10. The delivery system as recited in claim 7, further comprising a power source, a reservoir of therapeutic agents, means for recharging said power source, and means for refilling said reservoir of therapeutic agents.

11. The delivery system as recited in claim 7, further comprising means for affixing said system in the mouth of a living organism.

12. The delivery system as recited in claim 7, further comprising means for bidirectional information and command transfer to an external device.

13. The delivery system as recited in claim 7, further comprising means for monitoring the level of analyte by using near-infrared optical measurements.

14. The delivery system as recited in claim 7, further comprising means for monitoring the level of analyte by using a chemically treated semiconductor sensor.

15. The delivery system as recited in claim 2, further comprising a second capillary chamber in contact with said first capillary chamber.

16. The delivery system as recited in claim 15, wherein said second capillary chamber comprises a port disposed therein.

17. The delivery system as recited in claim 16, wherein said second capillary chamber is a radial distribution capillary chamber, said radial distribution occurring from said port outwardly in a direction parallel to said interface.
